(19) United States
(12) Patent Application Publication
(10) Pub. No.: US 2012/0209236 A1
(43) Pub. Date: Aug. 16, 2012

(54) EXTRA TABS DIAPER

(76) Inventor: Kristen Savea, Pearl City, HI (US)

(21) Appl. No.: 13/026,227

(22) Filed: Feb. 12, 2011

(51) Int. Cl. A61F 13/62

(52) U.S. Cl. 604/385.01

(57) ABSTRACT

A diaper having an extra set of tabs that fold over the diaper closure tabs to prevent the closure tabs from becoming accidently undone or becoming undone by an infant. The extra tabs are folded in an accordion-like fashion before being deployed. The extra tabs have a smooth outer surface to prevent discomfort to a wearer and a Velcro lined inner surface that mates with a Velcro strip on the back of the diaper. The extra tabs are folded in an accordion-like fashion before being deployed to enable efficient packaging.

### FIELD OF THE INVENTION

The presently disclosed subject matter is directed towards a diaper having closure tabs covered by another set of tabs that protect the closure tabs.

### BACKGROUND OF THE INVENTION

A diaper is an absorbent material worn by those who are unable or unwilling to use a toilet to dispose of body waste. A diaper captures body waste and thus prevents it from dropping around others. While diapers are most closely associated with children, they are also used by the elderly and the infirm.

Traditionally, a diaper was a piece of cloth, usually cotton, which was worn around a child's excretory areas and held in place by safety pins. In more recent decades disposable diapers have become more commonplace. A disposable diaper is typically comprised of an absorbent pad sandwiched between sheets of nonwoven fabric. The diaper is configured such that the pad covers a wearer's excretory area and forms corners that wrap around the wearer to hold the diaper in place. Retaining the corners in place is usually accomplished using closure tabs, typically Velcro or glued fasteners, at the sides of the wearer.

While prior art diapers have proven highly successful, they do have problems with wearer retention. Often, either by accident or design, a diaper can become undone by undoing the closure tabs.

Therefore, a technique for preventing the closure tabs of a diaper from becoming undone by accident or by the actions of a wearer would be beneficial.

### BRIEF SUMMARY OF THE INVENTION

The principles of the present invention provide for diapers having closure tabs that can be covered by an extra set of tabs that prevent the closure tabs from becoming accidently undone or becoming undone by an infant. The extra tabs have a smooth outer surface to prevent discomfort to a wearer and a Velcro lined inner surface that mates with a Velcro strip on the back of the diaper. The extra tabs fold over the diaper's closure tabs. The extra tabs are folded in an accordion-like fashion before being deployed to enable efficient packaging of the extra tabs diaper.

### BRIEF DESCRIPTION OF THE DRAWINGS

The advantages and features of the present invention will become better understood with reference to the following detailed description and claims when taken in conjunction with the accompanying drawings, in which like elements are identified with like symbols, and in which:

FIG. 1 is an environmental perspective view of an extra tabs diaper 10 according to a preferred embodiment of the present invention;

FIG. 2A is a perspective front view of the extra tabs diaper 10 according to FIG. 1 wherein a pair of extra tabs 30a, 30b is partially deployed;

FIG. 2B is a perspective front view of the extra tabs diaper 10 according to FIG. 1 where the extra tabs 30a, 30b are totally deployed; and

FIG. 3 is a perspective rear view of the extra tabs diaper 10.

### DETAILED DESCRIPTION OF THE INVENTION

The presently disclosed subject matter now will be described more fully hereinafter with reference to the accompanying drawings in which an embodiment is shown. However, it should be understood that this invention may take many different forms and thus should not be construed as being limited to the specific embodiment set forth herein. Additionally, the terms “a” and “an” used herein do not denote a limitation of quantity, but rather denote the presence of at least one of the referenced items.

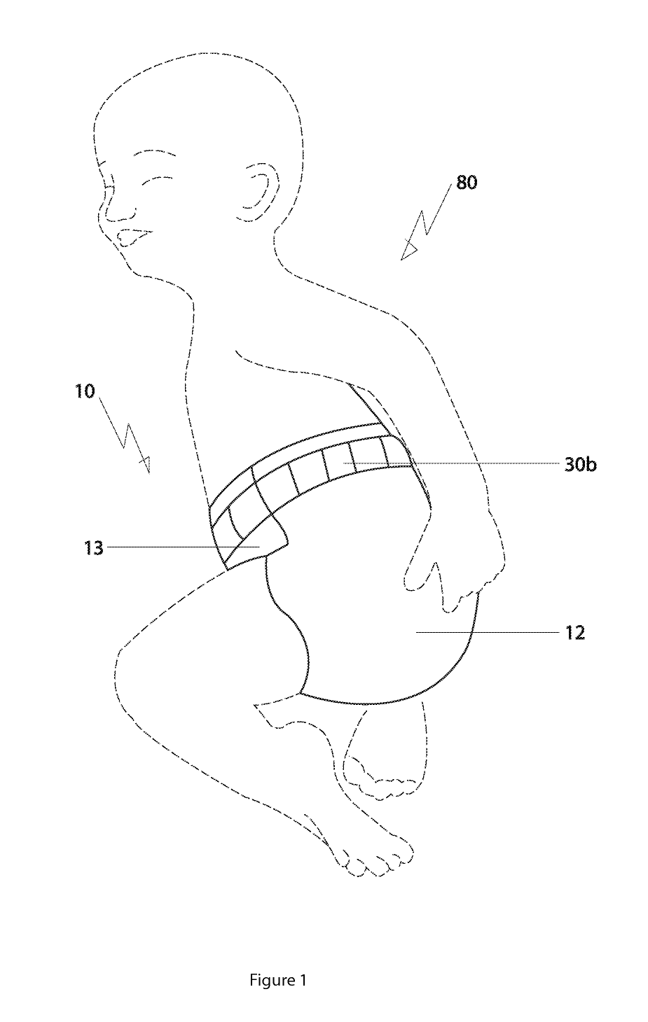

The principles of the present invention are illustrated in a specific embodiment of an extra tabs diaper 10, which is described herein with reference to FIGS. 1 to 3. The extra tabs diaper 10 includes a base diaper 12 which is akin to those found in the prior art. However, that base diaper 12 includes a pair of extra tabs 30a, 30b (only tab 30b being shown in FIG. 1, see other figures) that assist retention of the extra tabs diaper 10 on a wearer by reducing or eliminating the likelihood of the extra tabs diaper 10 becoming accidently undone or becoming undone by an infant.

FIG. 1 illustrates an extra tabs diaper 10 being worn by an infant 80. While an infant is shown it should be understood that the principles of the present invention are applicable to diapers worn by the elderly and the infirm. The extra tabs diaper 10 includes the base diaper 12 which has four corners 13 (only one of which is shown in FIG. 1, see other figures). FIG. 1 shows an extra tab 30b that extends from the front of the infant 80, around the side of the infant 80, and along the back of the infant 80.

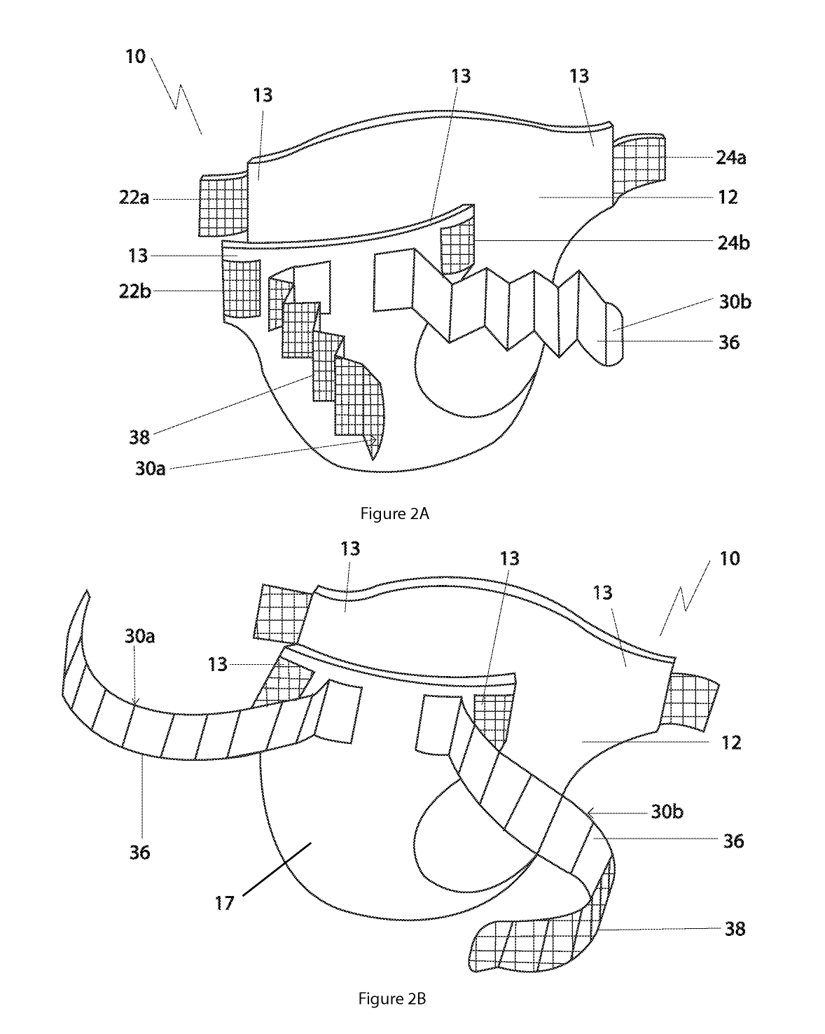

FIG. 2A illustrates a front perspective view of the extra tabs diaper 10. FIG. 2A shows the base diaper 12 as having a first back closure tab 22a located on a back corner 13 and a mating first front closure tab 22b that is located on a mating front corner 13 of the base diaper 12. Additionally, the base diaper 12 includes a second back closure tab 24a on another back corner 13 of the base diaper 12 and a mating second front closure tab 24b that is located on a mating front corner 13 of the base diaper 12. When the extra tabs diaper 10 is placed on a user such as an infant 80 the first front closure tab 22b is mated with the first back closure tab 22a to retain one side closed while the second front closure tab 24b is mated with the second back closure tab 24a to retain the other side closed. The first front closure tab 22b, the first back closure tab 22a, the second front closure tab 24b, and the second back closure tab 24a may be Velcro or glue strips or some other type of closure mechanism.

Referring now to FIGS. 2A and 2B, the base diaper 12 includes an additional pair of tabs, specifically the extra tab 30a and the extra tab 30b. As shown each extra tab 30a and 30b has a smooth outer surface 36 and a Velcro lined inner surface 38. Those extra tabs 30a, 30b are permanently fastened onto a front portion of the base diaper 12 by stitching or chemical bonding. The extra tab 30a is located so that when it wraps around a wearer that it covers a closed first front closure tab 22b and first back closure tab 22a. Similarly, the extra tab 30b is located so that when it is wraps around a wearer that it covers a closed second front closure tab 24b and second back closure tab 24a.

Turning now specifically to FIG. 2A, before deployment the extra tabs 30a and 30b are folded in an accordion-like fashion to allow each of the extra tabs 30a, 30b to be collapsed into a flat configuration for packaging. This allows a plurality of extra tabs diapers 10 to be compactly packaged without major interference by the extra tabs 30a and 30b. Furthermore, the accordion-like folding is performed such that the Velcro lined inner surface 38 is protected and does not contact itself.

Turning now specifically to FIG. 2B, when being deployed the extra tabs 30a and 30b are pulled from their accordion-like fold. This results in the smooth outer surface 36 being on the outside of the base diaper 12 and the Velcro lined inner surface 38 facing the base diaper 12. The extra tabs 30a and 30b are sufficiently long that when they are fully deployed on an infant 80 they wrap around both sides of the infant and at least part of the back of the infant 80. Furthermore, the extra tabs 30a and 30b are located such that when they wrap around an infant that they cover the closure tabs 22a, 22b, 24a, and 24b. The purpose of this is to prevent accidental damage to and user access to the closure tabs 22a, 22b, 24a, and 24b.

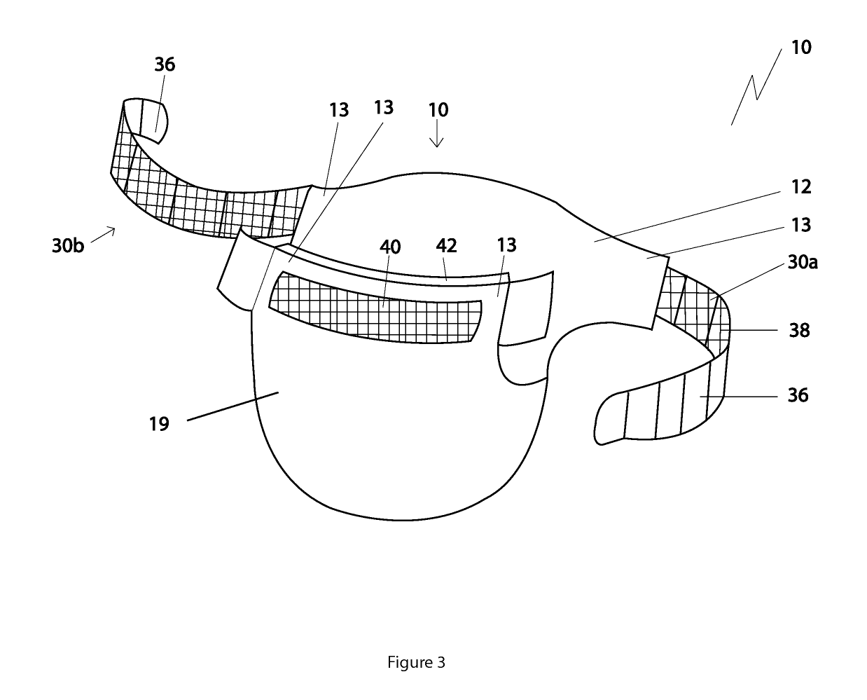

Referring now to FIG. 3, the extra tabs 30a and 30b are retained in position by mating with a rear Velcro strip 40. Ideally the Velcro strip 40 and the Velcro lined inner surfaces 38 are located such that they are prevented from directly contacting an infant. To assist this, Velcro strip 40 is located sufficiently below the top edge 42 of the base diaper 12 that Velcro strip 40 cannot contact the infant 80.

It is to be understood that while the figures and the above description illustrate the principles of the present invention, they are exemplary only. They are not intended to be exhaustive or to limit the invention to the precise forms disclosed, and obviously many modifications and variations are possible in light of the above teaching. Therefore, the present invention is to be limited only by the appended claims.

What is claimed:

1. A diaper, comprising:
a base diaper having a front, a rear having a top, and four corners;
a first set of closure tabs for closing a first set of two corners of said four corners;
a second set of closure tabs for closing a second set of two corners of said four corners;
a Velcro strip on said rear;
a first extra tab attached to said front, said first extra tab being located and sufficiently long to span across said first set of closure tabs to said Velcro strip, said first extra tab having a smooth outer surface and a Velcro lined inner surface; and
a second extra tab attached to said front, said second extra tab being located and sufficiently long to span across said second set of closure tabs to said Velcro strip, said second extra tab having a smooth outer surface and a Velcro lined inner surface.

2. A diaper according to claim 1, wherein when said first extra tab is not deployed said smooth outer surface contacts said Velcro lined inner surface such that said Velcro lined inner surface does not contact itself

3. A diaper according to claim 2, wherein said first extra tab and said second extra tab fold in accordion-like fashion when not deployed.

4. A diaper according to claim 1, wherein said Velcro strip on said rear is sufficiently below said top so as not to contact a wearer.
